(19) United States
(12) Patent Application Publication
(10) Pub. No.: US 2022/0251080 A1
(43) Pub. Date: Aug. 11, 2022

(54) SUBSTANTIALLY PURE VENETOCLAX AND AMORPHOUS VENETOCLAX IN A FREE DRUG PARTICULAT E FORM

(71) Applicant: DR. REDDY'S LABORATORIES LIMITED, Hyderabad (IN)

(72) Inventors: Sekhar MUNASWAMY NARIYAM, Hyderabad (IN); Veera Babu KAGITA, Mudinepalli (IN); Sridhar VASAM, Kothagudem Village (IN); Siva Reddy MAKIREDDY, Guntur (IN); Sridhar CHAGANTI, Hyderabad (IN); Shiva Prasad KOYYADI, Hyderabad (IN); Pradeep Kumar TYAGI, KVRanga Reddy (IN); Rajeev Rehani BUDHDEV, Hyderabad (IN)

(21) Appl. No.: 17/613,402

(22) PCT Filed: Jun. 26, 2020
(86) PCT No.: PCT/IB2020/056052

§ 371 Date: Nov. 22, 2021

(30) Foreign Application Priority Data
Jun. 28, 2019 (IN) 201941025914

(51) Int. Cl. C07D 471/04

(52) U.S. Cl. CPC C07D 471/04

(57) ABSTRACT

The present invention provides substantially pure venetoclax, process for the preparation of substantially pure venetoclax and pharmaceutical formulation of substantially pure venetoclax. In another aspect present invention provides amorphous venetoclax in a free drug particulate form, process for the preparation of amorphous venetoclax in a free drug particulate form and pharmaceutical formulation of amorphous venetoclax in a free drug particulate form.

### FIELD OF THE INVENTION

The present invention provides substantially pure venetoclax and preparation thereof. In another aspect present invention provides amorphous venetoclax in a free drug particulate form and preparation thereof. BACKGROUND OF THE INVENTION AND DISCLOSURE OF PRIOR ART The drug compound having the adopted name “Venetoclax” has chemical name: 4-(4-{[2-(4-chlorophenyl)-4,4dimethylcyclohex-1-en-1-yl]methyl}piperazin-1-yl)-N-({3-nitro-4-[(tetrahydro-2H-pyran-4ylmethyl)amino]phenyl}sulfonyl)-2-(1H-pyrrolo[2,3 -1)]pyridin-5-yloxy)benzamide) as below.

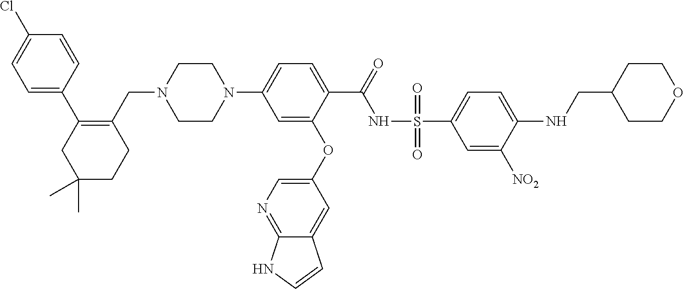 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax is a selective and orally bioavailable small-molecule inhibitor of BCL-2, an anti-apoptotic protein. Overexpression of BCL-2 has been demonstrated in CLL cells where it mediates tumor cell survival and has been associated with resistance to chemotherapeutics. Venetoclax helps restore the process of apoptosis by binding directly to the BCL-2 protein, displacing pro-apoptotic proteins like BIM, triggering mitochondrial outer membrane permeabilization and the activation of caspases. In nonclinical studies, Venetoclax has demonstrated cytotoxic activity in tumor cells that overexpress BCL-2.

Venetoclax is approved in US as VENCLEXTA tablet for oral administration for the treatment of patients with chronic lymphocytic leukemia with 17p deletion, as detected by an FDA approved test, who have received at least one prior therapy. This indication is approved under accelerated approval based on overall response rate. VENCLEXTA is available as 10, 50 and 100 mg tablets with dosage of 20 mg once daily for 7 days, followed by a weekly ramp-up dosing schedule to the recommended daily dose of 400 mg.

U.S. Pat. No. 8,546,399 B2 illustrates the usefulness of Venetoclax as an inhibitor of BCL-2 protein. Further, it discloses preparative methods for the preparation of compounds disclosed therein including Venetoclax by reacting 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-y1)benzoic acid with 3 -nitro-4-(((tetrahydro-2H-pyran-4-yl)methyl)amino)benzenesulfonamide. The product was isolated by chromatography from 25-100% ethyl acetate/hexane and then with 10% methanol/ethyl acetate with 1% acetic acid as a white solid.

U.S. Pat. No. 9,006,438 B2 describes an improved process for the preparation of Venetoclax through the formation of tert. Butyl 2-((1H-pyrrolo[2,3-b]pyridin-5-yl)oxy)-4-(4-(4′-chloro-5,5-dimethyl-3,4,5,6-tetrahydro-[1,1′-biphenyl]-2-yl)methyl) piperazin-1-yl)benzoate by reacting tert. butyl ester of 2-((1H-pyrrolo[2,3-b]pyridin-5-yl)oxy)-4-bromobenzoate with 1-((4′-chloro-5,5-dimethyl-3,4,5,6-tetrahydro-[1,1′-biphenyl]-2-yl)methyl)piperazine in the presence of tert. butoxide salt.

WO2019150253A1 describes a process for the purification of Venetoclax through the alkali metal salt formation, also discloses process for the preparation of an amorphous Venetoclax.

WO2017212431A1 and WO2018167652A1 describe processes for the preparation of amorphous form of Venetoclax by solvent and anti-solvent methods.

There is a continuing need in the art to provide a substantially pure venetoclax having reduced amounts of impurity as compared to known in the art. The present application relates to substantially pure venetoclax and preparation thereof. The present application also relates to amorphous venetoclax in a free drug particulate form and preparation thereof.

All references cited herein are incorporated by reference in their entireties for all purposes.

### SUMMARY OF THE INVENTION

In first embodiment, the present invention provides a substantially pure venetoclax. In one aspect of first embodiment, the substantially pure venetoclax comprises less than about 0.01% of N-oxide compound corresponding to Formula (A):

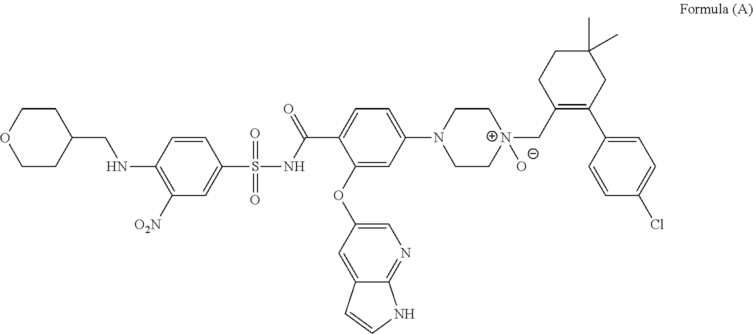 [SMILES: CC1(C)CCC(C[N+]2([O-])CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In another aspect of first embodiment, the present invention provides substantially pure venetoclax comprising less than about 0.008% of N-oxide compound.

In second embodiment, the present invention provides a process for the preparation of substantially pure venetoclax, the process comprising the steps of:
a) providing venetoclax in ether solvent; and
b) isolating the substantially pure venetoclax.

In third embodiment, the present invention provides a pharmaceutical formulation comprising substantially pure venetoclax, and pharmaceutically acceptable excipients.

In fourth embodiment, the present invention provides an amorphous venetoclax in a free drug particulate form. In one aspect of fourth embodiment, the amorphous venetoclax in a free drug particulate form has Carr's Index (%) less than 26.

In fifth embodiment, the present invention provides a process for the preparation of amorphous venetoclax in a free drug particulate form, the process comprising the steps of:
a) providing venetoclax in glacial acetic acid;
b) treating with ammonia solution; and
c) isolating the amorphous form of venetoclax in a free drug particulate form.

In sixth embodiment, the present invention provides a pharmaceutical formulation comprising amorphous venetoclax in a free drug particulate form, and a pharmaceutically acceptable excipient.

In seventh embodiment, the present invention provides a process for the preparation of Venetoclax.

### BRIEF DESCRIPTION OF THE DRAWINGS

FIG. 1 illustrates a powder X-Ray Diffraction pattern of amorphous Venetoclax in a free drug particulate form, obtained in Example 6.

FIG. 2 illustrates a HPLC chromatogram of substantially pure venetoclax, obtained in Example 4.

FIG. 3 illustrates a HPLC chromatogram of venetoclax, obtained from Innovator RLD

### DETAILED DESCRIPTION

In first embodiment, the present invention provides substantially pure venetoclax.

In an aspect of first embodiment, the present invention provides substantially pure venetoclax comprising less than about 0.01% of N-oxide compound corresponding to Formula (A):

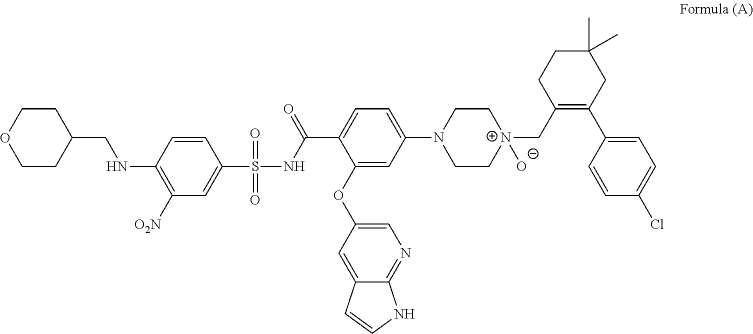 [SMILES: CC1(C)CCC(C[N+]2([O-])CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In another aspect of first embodiment, the present invention provides substantially pure venetoclax comprising less than about 0.008% of N-oxide compound.

In another aspect of first embodiment, the present invention provides the substantially pure venetoclax, characterized by HPLC chromatogram, substantially as depicted in FIG. 2

In second embodiment, the present invention provides a process for the preparation of substantially pure venetoclax, the process comprising the steps of:
a) providing venetoclax in ether solvent; and
b) isolating the substantially pure venetoclax.

In an aspect of second embodiment, the purification process is optionally repeated to give the substantially pure venetoclax.

Suitable ether solvent which can be used in step (a) is selected from the group consisting of C₂₋₈ alkyl ethers, such as 1,4-dioxane, tetrahydrofuran or mixtures thereof. Most preferred ether solvent is 1,4-dioxane.

In an aspect of second embodiment, ether solvent can be removed by any known method in the art, such as evaporation.

In third embodiment, the present invention provides a pharmaceutical formulation comprising substantially pure venetoclax, and pharmaceutically acceptable excipients.

Suitable pharmaceutically acceptable excipient can be selected from the group consisting of polyvinyl pyrrolidone, povidone K-30, povidone K-60, Povidone K-90, polyvinylpyrrolidone vinylacetate, co-povidone NF, polyvinylacetal diethylamino acetate (AEA®), polyvinyl acetate phthalate, polysorbate 80, polyoxyethylene-polyoxypropyl ene copolymers (Poloxamer® 188), polyoxyethylene (40) stearate, polyethyene glycol monomethyl ether, polyethyene glycol, poloxamer 188, pluronic F-68, methylcellulose, methacrylic acid copolymer (Eudragit or Eudragit-RLPO), hydroxypropylmethyl cellulose phthalate, hydroxypropylmethyl cellulose acetate succinate (HPMC-AS), hydroxypropylmethyl cellulose, hydroxypropyl cellulose SSL(HPC-SSL), hydroxypropyl cellulose SL(HPC-SL), hydroxypropyl cellulose L (HPC-L), hydroxyethyl cellulose, Soluplus® (polyvinyl capro lactam-polyvinyl acetate-polyethylene glycol graft copolymer (PCL-PVAc-PEG)), gelucire 44/14, ethyl cellulose, D-alpha-tocopheryl polyethylene glycol 1000 succinate, cellulose acetate phthalate, carboxymethylethylcellulose and the like; cyclodextrins, gelatins, hypromellose phthalates, sugars, polyhydric alcohols, and the like.

In an aspect of present invention, the substantially pure venetoclax according to the present invention have reduced amounts of N-oxide impurity as compared to Ventoclax API (isolated from Venclexta).

In fourth embodiment, the present invention provides amorphous venetoclax in a free drug particulate form. In another aspect of fourth embodiment, the amorphous venetoclax in a free drug particulate form having carr's index (%) less than 26.

In fifth embodiment, the present invention provides a process for the preparation of amorphous venetoclax in a free drug particulate form, the process comprising the steps of:
a) providing venetoclax in glacial acetic acid;
b) treating with ammonia solution; and
c) isolating amorphous form of venetoclax in a free drug particulate form.

In another aspect of fifth embodiment, the amorphous form of Venetoclax may be isolated by employing any of the techniques, but not limited to: filtration by gravity or suction, centrifugation, adding solvent to make slurry followed by filtration or decantation and optionally washing with water.

Amorphous venetoclax obtained from the present process shows improved physical properties w.r.t flow of the material and filtration operation due to aggregation phenomena. As per the flow property measurement, powder obtained by reactive agglomeration has better flow indices (Carr's index and Hausner ratio) than powder obtained by solvent-antisolvent process, disclosed in example 24 of WO2017212431. Filtration characteristics studied for both processes (i.e., solvent-anti solvent and reactive agglomeration), the present invention reactive agglomeration process shows a significant reduction in the specific cake resistance, improve filtration operation and thereby reducing filtration time.

### Comparison Data:

| | Amorphous venetoclax | Amorphous venetoclax |
|---|---|---|
| | obtained by Solvent- | obtained by reactive |
| | Anti solvent Process | agglomeration process |
| Property | (Prior Art) | (Present Invention) |
| Bulk density (g/ml) | 0.203 | 0.328 |
| Tapped density (g/ml) | 0.289 | 0.442 |
| Hausner ratio | 1.4 | 1.3 |
| Carr's index | 29.8 | 25.8 |
| Specific cake | Filter media clogged | 2.73E+10 |
| resistance (m/Kg) | | |

In sixth embodiment, the present invention provides a pharmaceutical formulation comprising amorphous venetoclax in a free drug particulate form, and pharmaceutically acceptable excipients.

Suitable pharmaceutically acceptable excipients are selected from the group consisting of polyvinyl pyrrolidone, povidone K-30, povidone K-60, Povidone K-90, polyvinylpyrrolidone vinylacetate, co-povidone NF, polyvinylacetal diethylamino acetate (AEA®), polyvinyl acetate phthalate, polysorbate 80, polyoxyethylene-polyoxypropylene copolymers (Poloxamer® 188), polyoxyethylene (40) stearate, polyethyene glycol monomethyl ether, polyethyene glycol, poloxamer 188, pluronic F-68, methylcellulose, methacrylic acid copolymer (Eudragit or Eudragit-RLPO), hydro xyethyl cellulose phthalate, hydroxypropylmethyl cellulose acetate succinate (HPMC-AS), hydroxypropylmethyl cellulose, hydroxypropyl cellulose SSL(HPC-SSL), hydroxypropyl cellulose SL(HPC-SL), hydroxypropyl cellulose L (HPC-L), hydro xyethyl cellulose, Soluplus® (polyvinyl capro lactam-polyvinyl acetate-polyethylene glycol graft copolymer (PCL-PVAc-PEG)), gelucire 44/14, ethyl cellulose, D-alpha-tocopheryl polyethylene glycol 1000 succinate, cellulose acetate phthalate, carboxymethylethylcellulose and the like; cyclodextrins, gelatins, hypromellose phthalates, sugars, polyhydric alcohols, and the like. In seventh embodiment, the present invention provides a process for the preparation of Venetoclax. In an aspect, the process for the preparation of Venetoclax can be illustrated by Scheme-3:

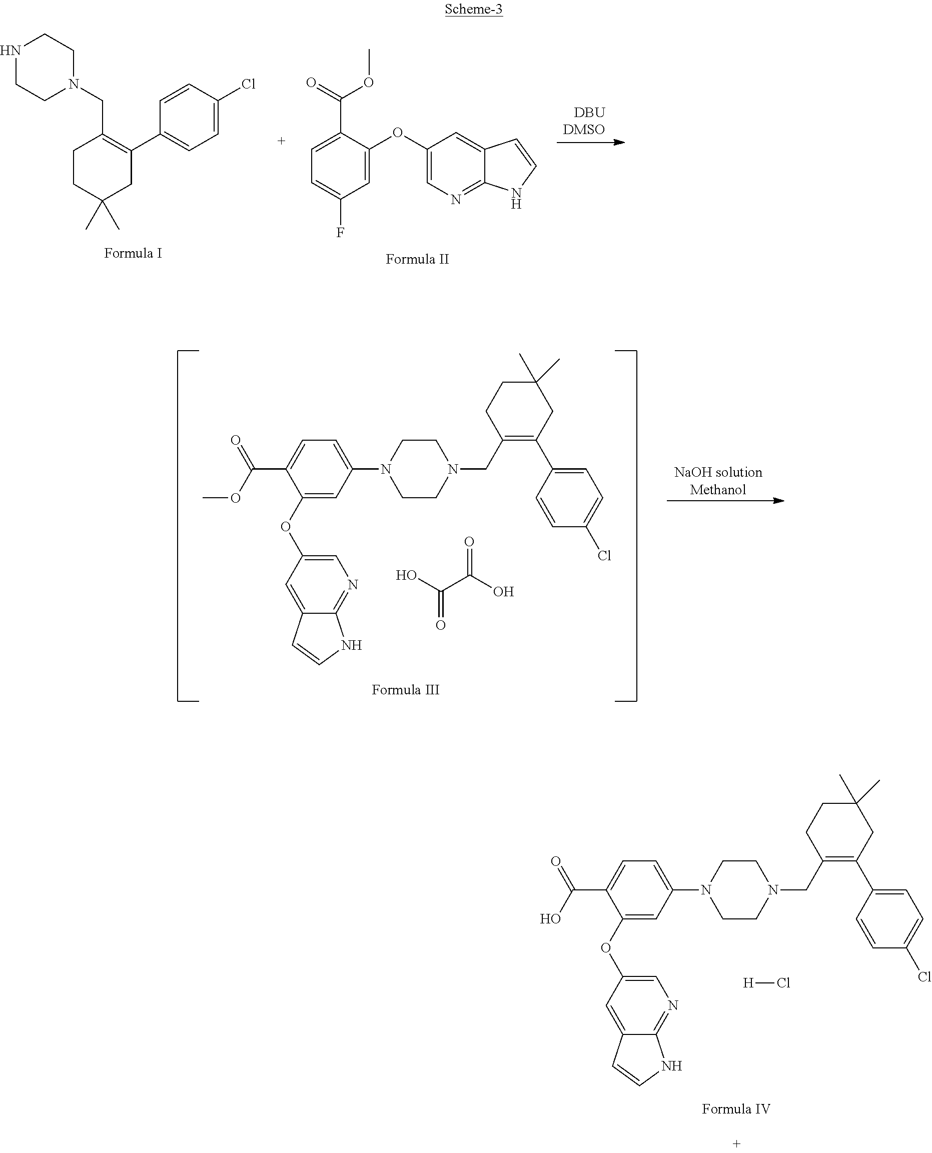

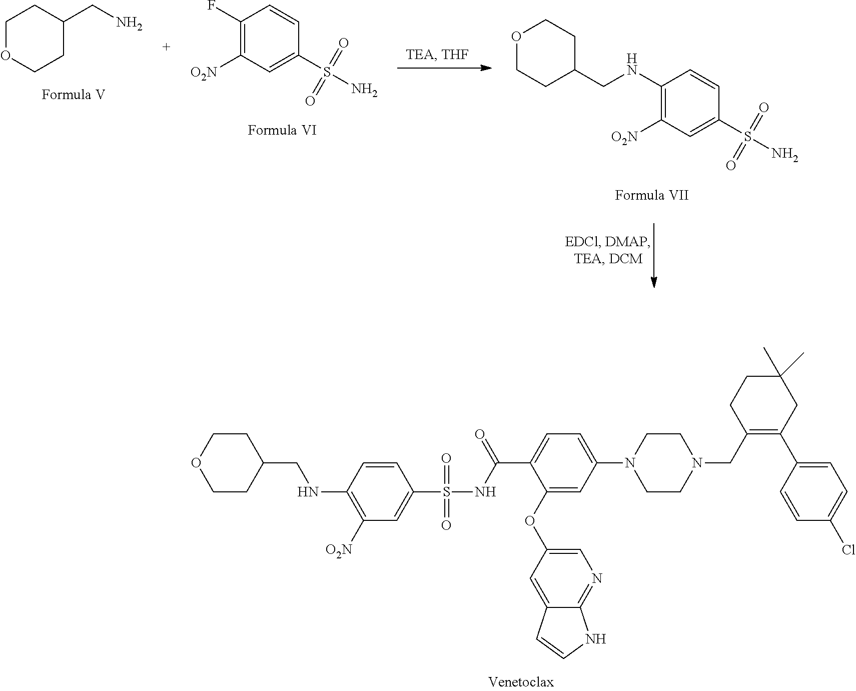 [SMILES: NCC1CCOCC1 ; NS(=O)(=O)c1ccc(F)c([N+](=O)[O-])c1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

The starting materials (formula I, II and VII) of Venetoclax can be prepared by any known method or by the process that is illustrated as given below in scheme 4, 5, 6, 7, 8 and 9.

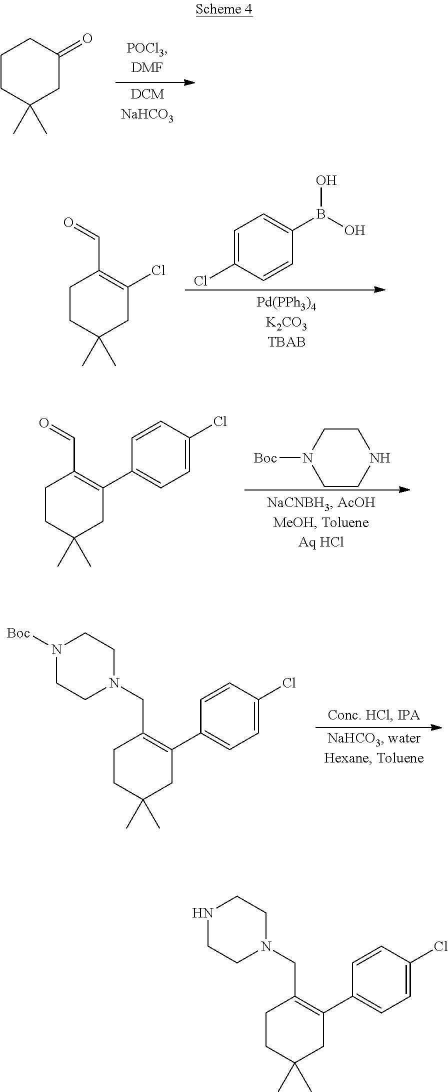

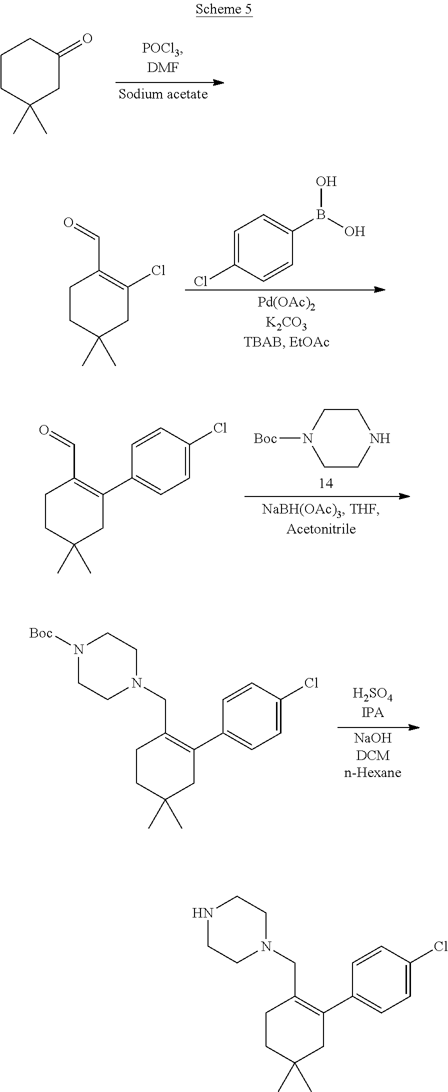

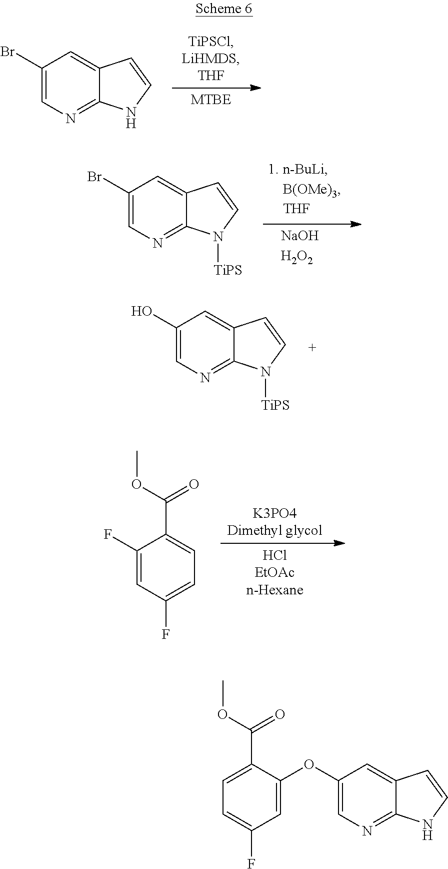

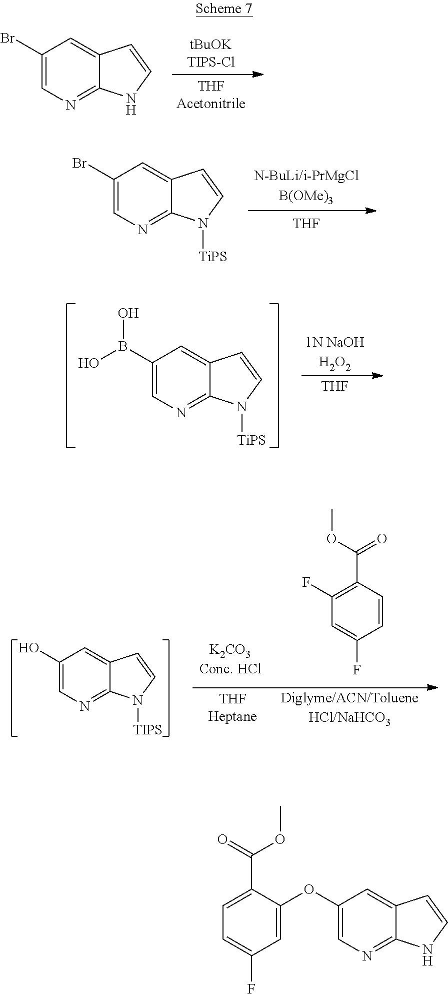

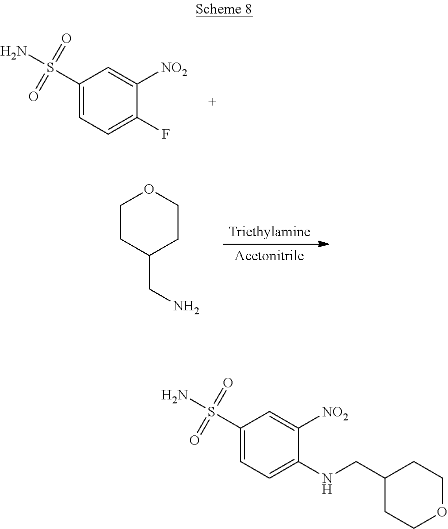 [SMILES: NS(=O)(=O)c1ccc(F)c([N+](=O)[O-])c1 ; NCC1CCOCC1 ; NS(=O)(=O)c1ccc(NCC2CCOCC2)c([N+](=O)[O-])c1]

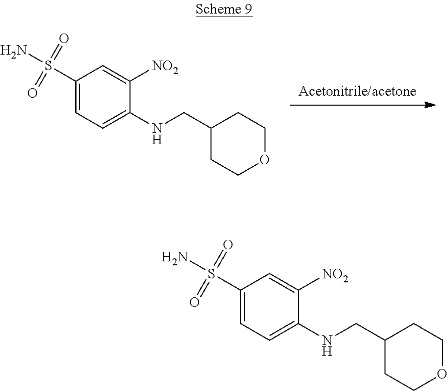 [SMILES: NS(=O)(=O)c1ccc(NCC2CCOCC2)c([N+](=O)[O-])c1 ; NS(=O)(=O)c1ccc(NCC2CCOCC2)c([N+](=O)[O-])c1]

Venetoclax produced by the method of present invention can be chemically pure having purity greater than about 99.5% and containing no single impurity in amounts greater than about 0.15%, by HPLC.

In an aspect, Venetoclax prepared according to the present invention synthetic process may have following possible impurities:

| No. | IUPAC name of Impurity | Structure |
|---|---|---|
| 1. | 2-((1H-Pyrrolo[2,3-b]pyridin-5-yl)oxy)-4-(4- ((4′-chloro-5,5-dimethyl-3,4,5,6-tetrahydro- [1,1′-biphenyl]-2-yl)methyl)piperazin-1- yl)benzoic acid Hydrochloride | 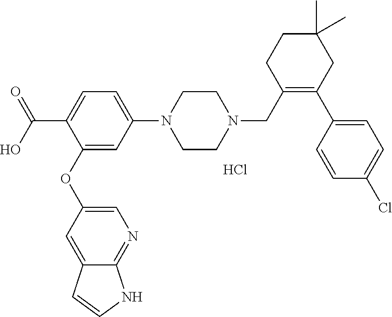 [SMILES: CC1(C)CCC(CN2CCN(c3ccc(C(=O)O)c(Oc4cnc5[nH]ccc5c4)c3)CC2)=C(c2ccc(Cl)cc2)C1 ; Cl] |
| 2. | 3-Nitro-4-(((tetrahydro-2H-pyran-4- yl)methyl)amino)benzenesulfonamide | 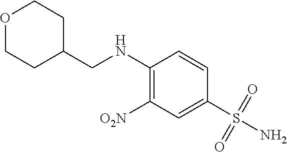 [SMILES: NS(=O)(=O)c1ccc(NCC2CCOCC2)c([N+](=O)[O-])c1] |
| 3. | N1,N2-bis((3-nitro-4-(((tetrahydro-2H-pyran- 4-yl)methyl)amino)phenyl)sulfonyl) oxalamide | 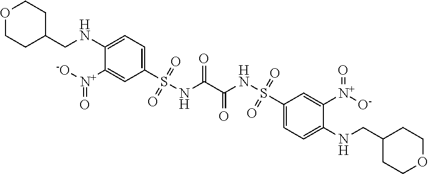 |
| 4. | 2-((1H-pyrrolo[2,3-b]pyridin-5-yl)oxy)-4- fluoro-N-((3-nitro-4-(((tetrahydro-2H-pyran- 4-yl)methyl)amino)phenyl) sulfonyl)benzamide | 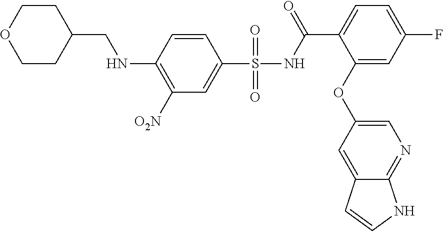 [SMILES: O=C(NS(=O)(=O)c1ccc(NCC2CCOCC2)c([N+](=O)[O-])c1)c1ccc(F)cc1Oc1cnc2[nH]ccc2c1] |
| 5. | 2-((1H-pyrrolo[2,3-b]pyridin-5-yl)oxy)-4-(4- ((5,5-dimethyl-3,4,5,6-tetrahydro-[1,1′- biphenyl]-2-yl)methyl)piperazin-l-yl)-N-((3- nitro-4-(((tetrahydro-2H-pyran-4- yl)methyl)amino)phenyl)sulfonyl)benzamide | 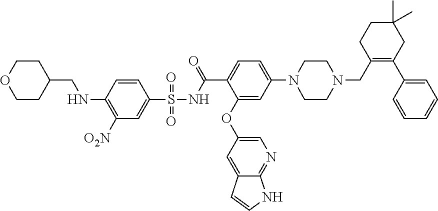 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccccc2)C1] |
| 6. | 2-((1-acetyl-1H-pyrrolo[2,3-b]pyridin-5- yl)oxy)-4-(4-((4′-chloro-5,5-dimethyl-3,4,5,6- tetrahydro-[1,1′-biphenyl]-2- yl)methyl)piperazin-l-yl)-N-((3-nitro-4- (((tetrahydro-2H-pyran-4-yl)methyl)amino) phenyl)sulfonyl)benzamide | 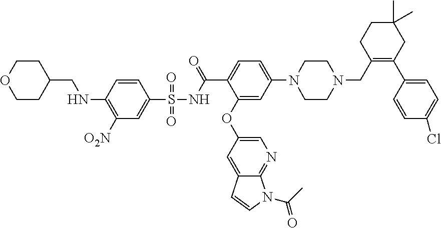 [SMILES: CC(=O)n1ccc2cc(Oc3cc(N4CCN(CC5=C(c6ccc(Cl)cc6)CC(C)(C)CC5)CC4)ccc3C(=O)NS(=O)(=O)c3ccc(NCC4CCOCC4)c([N+](=O)[O-])c3)cnc21] |
| 7. | 2-((1H-pyrrolo[2,3-b]pyridin-5-yl)oxy)-N-(4- (N-(2-((1H-pyrrolo[2,3-b]pyridin-5-yl)oxy)- 4-(4-((4′-chloro-5,5-dimethyl-3,4,5,6- tetrahydro-[1,1′-biphenyl]-2- yl)methyl)piperazin-1-yl)benzoyl) sulfamoyl)-2-nitrophenyl)-4-(4-((4′-chloro- 5,5-dimethyl-3,4,5,6-tetrahydro-[1,1′- biphenyl]-2-yl)methyl)piperazin-1-yl)-N- ((tetrahydro-2H-pyran-4-yl)methyl) benzamide | 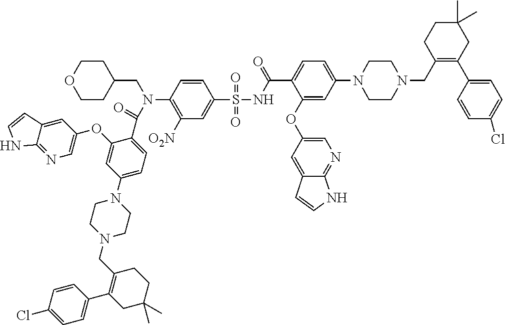 [SMILES: CC1(C)CCC(CN2CCN(c3ccc(C(=O)NS(=O)(=O)c4ccc(N(CC5CCOCC5)C(=O)c5ccc(N6CCN(CC7=C(c8ccc(Cl)cc8)CC(C)(C)CC7)CC6)cc5Oc5cnc6[nH]ccc6c5)c([N+](=O)[O-])c4)c(Oc4cnc5[nH]ccc5c4)c3)CC2)=C(c2ccc(Cl)cc2)C1] |
| 8. | 2-((1H-pyrrolo[2,3-b]pyridin-5-yl)oxy)-4-(5- (5-(4-((4′-chloro-5,5-dimethyl-3,4,5,6- tetrahydro-[1,1′-biphenyl]-2-yl)methyl) piperazin-1-yl)-2-(((3-nitro-4-(((tetrahydro- 2H-pyran-4-yl)methyl)amino)phenyl) sulfonyl) carbamoyl) phenoxy)-1H- pyrrolo[2,3-b]pyridin-1-yl)-N-((3-nitro-4- (((tetrahydro-2H-pyran-4-yl)methyl)amino) phenyl)sulfonyl)benzamide | 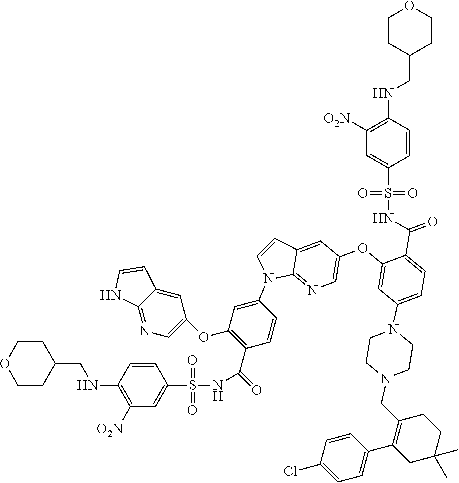 [SMILES: CC1(C)CCC(CN2CCN(c3ccc(C(=O)NS(=O)(=O)c4ccc(NCC5CCOCC5)c([N+](=O)[O-])c4)c(Oc4cnc5c(ccn5-c5ccc(C(=O)NS(=O)(=O)c6ccc(NCC7CCOCC7)c([N+](=O)[O-])c6)c(Oc6cnc7[nH]ccc7c6)c5)c4)c3)CC2)=C(c2ccc(Cl)cc2)C1] |
| 9. | N-(1-(4-(N-(2-((1H-pyrrolo[2,3-b]pyridin-5- yl)oxy)-4-(4-((4+40-chloro-5,5-dimethyl-3,4,5,6- tetrahydro-[1,1′-biphenyl]-2-yl)methyl) piperazin-1-yl)benzoyl) sulfamoyl)-2- nitrophenyl)pyridin-4(1H)-ylidene)-N- methylmethanaminium | 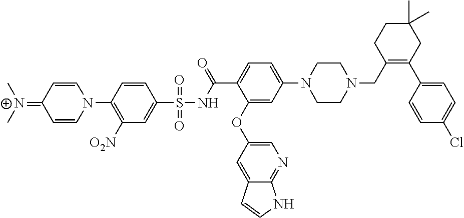 [SMILES: C[N+](C)=c1ccn(-c2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2[N+](=O)[O-])cc1] |
| 10. | 2-((1H-pyrrolo[2,3-b]pyridin-5-yl)oxy)-4-(4- ((4′-chloro-6-hydroxy-5,5-dimethyl-3,4,5,6- tetrahydro-[1,1′-biphenyl]-2- yl)methyl)piperazin-1-yl)-N-((3-nitro-4- (((tetrahydro-2H-pyran-4- yl)methyl)amino)phenyl)sulfonyl)benzamide | 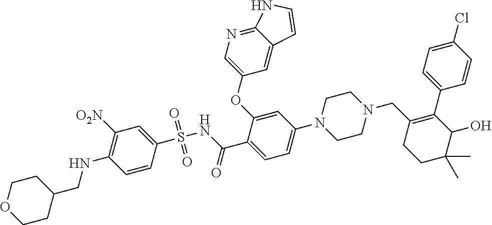 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1O] |
| 11 | 1,1′-((((5,5,5′′′, 5′′′-tetramethyl- 3,3′′′,4,4′′′,5,5′′′,6,6′′′-octahydro- [1,1′:4′,1″:4″,1′′′-quaterphenyl]-2,2′′′- diyl)bis(methylene))bis(piperazine-4,1- diyl))bis(2-((1H-pyrrolo[2,3-b]pyridin-5- yl)oxy)-4,1-phenylene))bis(N-((3-nitro-4- (((tetrahydro-2H-pyran-4- yl)methyl)amino)phenyl)sulfonyl)formamide) | 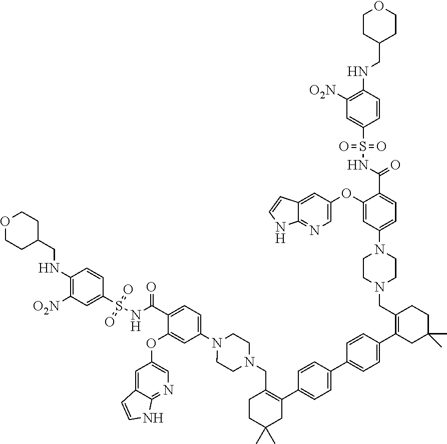 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(-c3ccc(C4=C(CN5CCN(c6ccc(C(=O)NS(=O)(=O)c7ccc(NCC8CCOCC8)c([N+](=O)[O-])c7)c(Oc7cnc8[nH]ccc8c7)c6)CC5)CCC(C)(C)C4)cc3)cc2)C1] |

The possible impurities mentioned above are found to be less than 0.15% in the Venetoclax produced according to the process of the present application. Venetoclax and its impurities can be analyzed using high performance liquid chromatography (HPLC), such as with a liquid chromatograph equipped with variable wavelength UV-detector and the method described below:

| Column | YMC ODS-A 150 × 4.6 mm, 3.01 μm | |
|---|---|---|
| Wavelength | 235 | nm |
| Flow rate | 1.0 | mL/minute |
| Temperature | 40° C. | |
| Concentration | 0.5 | mg/mL |
| Injection volume | 10 | mL |

| | |
|---|---|
| Diluent 1 | Acetonitrile |
| Diluent 2 | 0.1% Orthophosphoric acid: Acetonitrile (1:1 % v/v) |
| Elution | Gradient |

Mobile phase-A: Mix Buffer and Acetonitrile in the ratio of 700:300.
Mobile phase-B: Mix Buffer and Acetonitrile in the ratio of 300:700.
Buffer: Transfer accurately 10.0 mL of Triethylamine in 2000 mL
Milli-Q-Water, adjust the pH to 2.2 ± 0.05 with
Orthophosphoric acid (88%)
Gradient program:

| Time | % Mobile phase-A | % Mobile phase-B |
|---|---|---|
| 0.0 | 90 | 10 |
| 5 | 90 | 10 |
| 38 | 10 | 90 |
| 45 | 10 | 90 |
| 46 | 90 | 10 |
| 55 | 90 | 10 |

### Definitions

The term “substantially pure venetoclax” as used herein refers to venetoclax having purity greater than about 99% and free from N-oxide impurity. In an embodiment, the term “free from N-oxide impurity” means that N-oxide impurity is less than 0.01% (w/w) or less than 0.008% (w/w) in pure venetoclax. The term “free drug” refers to solid particles not intimately embedded in a coprecipitate.

The term “particulate” refers to one or more individual particles.

The term “bulk density” as used herein refers to the ratio of the mass to the volume (including the interparticulate void volume) of an untapped powder sample.

The term “tapped density” as used herein and it is obtained by mechanically tapping a graduated cylinder containing the sample until little further volume change is observed.

The tapping can be performed using different methods. The tapped density is calculated as mass divided by the final volume of the powder.

The term “carr's index” as used herein and it is calculated using below formula Carr Index =(tapped density-bulk density)/tapped density×100.

The term “hausner ratio” as used herein and it is calculated using below formula Hausner Ratio =Bulk density/Tapped density

The term “specific cake resistance” as used herein refers to filter cake is formed by the substances that are retained on a filter. The filter cake grows in the course of filtration, becoming thicker as particulate matter is retained. With increasing layer thickness, the flow resistance of the filter cake increases. Specific cake resistance is a measure of this resistance offered by cake towards the flow.

### EXAMPLES

### Example-1

### Preparation of methyl 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl) piperazin-1-yl)benzoate oxalate (Formula III)

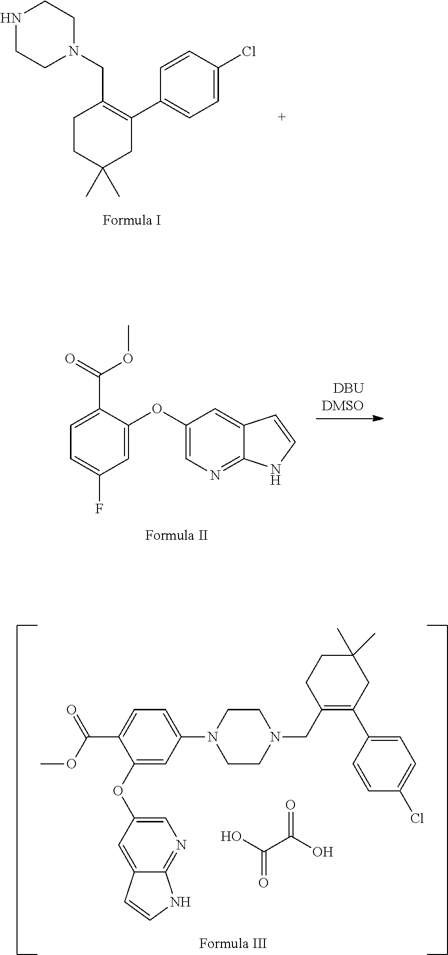

1-((4′-chloro-5,5-dimethyl-3,4,5,6-tetrahydro-[1,1′-biphenyl]-2-yl)methyl)piperazine (468 g) and toluene (4500 mL) and water (4500 mL) were charged into a round bottom flask at 25° C. The reaction mass was stirred for 15 minutes. The resulted organic layer and aqueous layers were separated. The organic layer was washed with water (9000 mL). The organic layer was evaporated under vacuum at 55° C. The resulted reaction mass, methyl 2-((1H-pyrrolo[2,3-b]pyridin-5-yl)oxy)-4-fluorobenzoate (300 g), DBU (160 g) and DMSO (900 mL) were charged into chemglass reactor at 22° C. The reaction mass was stirred for 68 hrs at 67° C. The reaction mass cooled to 23° C. The resulted reaction mass, ethyl acetate (6000 mL) and water (6000 mL) were charged into a round bottom flask at 25° C. The reaction mass was stirred for 15 minutes. The resulted organic layer and aqueous layers were separated. The organic layer, methanol (1500 mL) and oxalic acid dihydrate (330 g) were charged into a round bottom flask at 25° C. The reaction mass was stirred for 4 hrs at 29° C. The reaction mass was filtered under vacuum and washed with methanol (1500 mL). The solid was suck dried for 2 hrs. The solid was dried under vacuum at 60° C. Product weight: 811.6 g

### Example-2

### Preparation of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid hydrochloride (Formula IV)

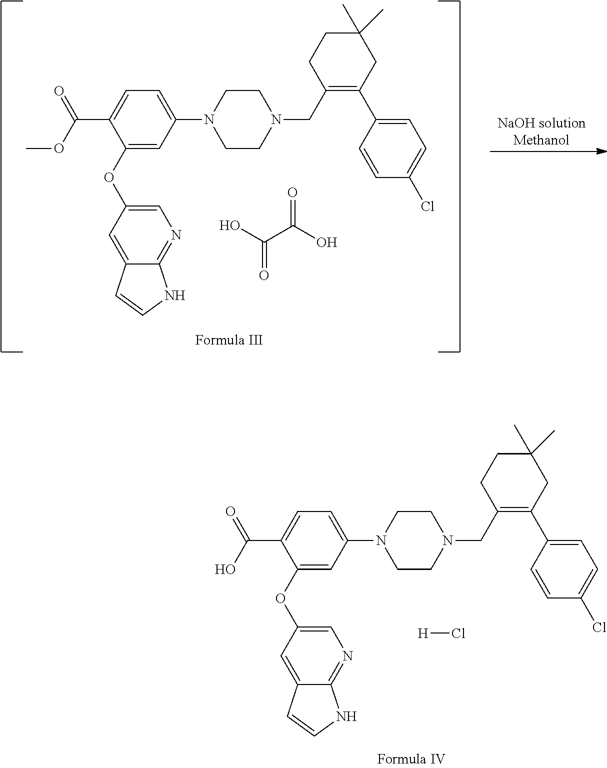

Water (513 mL) and sodium hydroxide (91 g) were charged into a round bottom flask at 25° C. The reaction mass was stirred for 15 minutes. Methyl 2-((1H-pyrrolo[2,3-b]pyridin-5 -yl)oxy)-4-(4-((4′-chloro-5, 5 -dimethyl-3,4,5,6-tetrahydro-[1,1′-biphenyl]-2-yl)methyl) piperazin-1-yl)benzoate oxalate (205 g) and methanol (1640 mL) were added to the reaction mass at 36° C. The reaction mass heated to 65° C. and stirred for 11 hrs. The reaction mass cooled to 26° C. Water (3075 mL) was added to the reaction mass at 26° C. The reaction mass cooled to 12° C. 2N HCl (1230 mL) was added to the reaction mass at 12° C. The reaction mass was stirred for 15 hrs at 25° C. The reaction mass was filtered and washed with water (4100 mL). The solid was suck dried for 2 hrs. The solid was dried under vacuum at 58° C. Product weight: 113.8 g; Purity by HPLC: 95.52%.

### Example-3

### Preparation of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid hydrochloride (Formula IV)

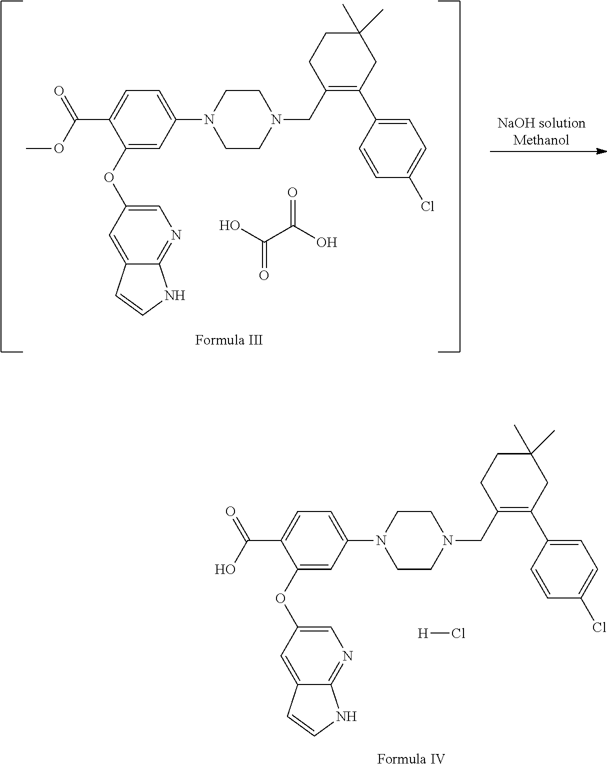

Methyl 2-41H-pyrrolo[2,3-b]pyridin-5-yl)oxy)-4-(4-((4′-chloro-5,5-dimethyl-3,4,5,6-tetrahydro-[1,1′-biphenyl]-2-yl)methyl) piperazin-l-yl)benzoate oxalate (284 g) and methanol (2272 mL) were charged into a round bottom flask at 26° C. The reaction mass was stirred for 10 minutes. Aqueous sodium hydroxide solution (126 g in 710 mL of Water) was added to the reaction mass at 26° C. The reaction mass heated to 65° C. and stirred for 10 hrs. The reaction mass cooled to 15° C. Water (4260 mL) was added to the reaction mass at 15° C. The reaction mass cooled to 13° C. 2N HCl (1704 mL) was added to the reaction mass at 13° C. The reaction mass was stirred for 40 minutes at 13° C. The reaction mass was stirred for 3 hrs at 21° C. The reaction mass was filtered and washed with water (5680 mL). The solid was suck dried for 2 hrs. The solid was dried under vacuum oven at 61° C. Product weight: 180.7 g.

2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid hydrochloride (79 g) and DMF (1253 mL) were charged into round bottom flask at 23° C. The reaction mass was stirred was stirred for 10 minutes. The reaction mass was heated to 73° C. and stirred for 2 hrs 30 minutes. The reaction mas cooled to 46° C. and stirred for 2 hrs 30 minutes. The reaction mass was filtered and washed with DMF (358 mL) followed by water (3580 mL). The solid was suck dried for 2 hrs. The solid was dried under vacuum oven at 61° C. Product weight:140.5 g.

### Example-4

### Preparation of Venetoclax

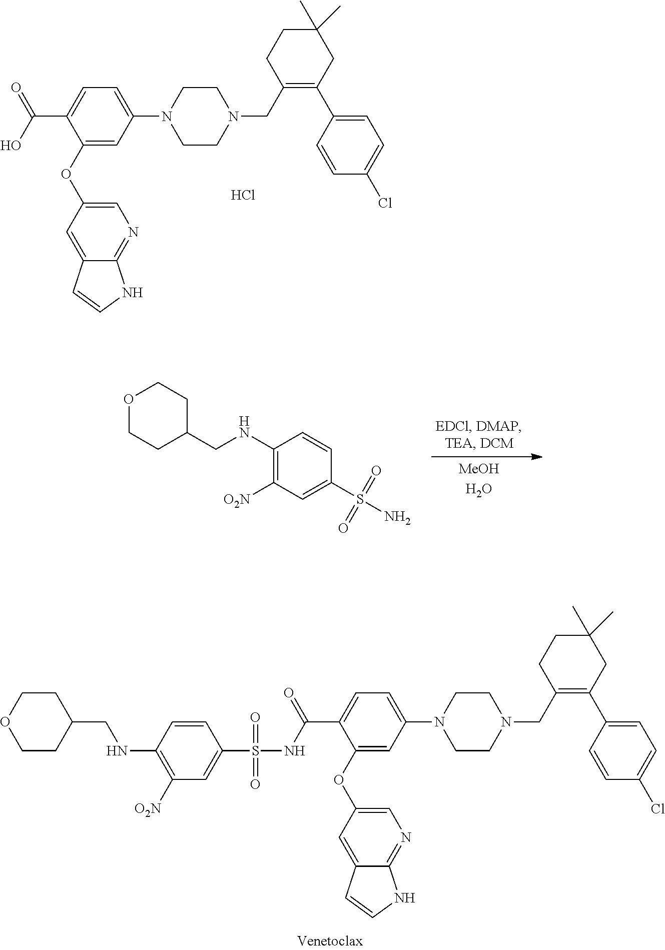 [SMILES: CC1(C)CCC(CN2CCN(c3ccc(C(=O)O)c(Oc4cnc5[nH]ccc5c4)c3)CC2)=C(c2ccc(Cl)cc2)C1 ; Cl ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid hydrochloride (5 g), 3-nitro-4-(((tetrahydro-2H-pyran-4-yl)methyl)amino)benzenesulfonamide (2.85 g), EDC.HCl (2.366 g), DMAP (2.011 g), triethylamine (2.91 g), dichloromethane (125 mL) were charged into a round bottom flask at 27° C. The reaction mass was stirred for 11 hrs at 27° C. Water (100 mL) and dichloromethane (50 mL) were added to the reaction mass at 27° C. The reaction mass was stirred for 20 minutes at 27° C. The resulted organic layer and aqueous layers were separated. The organic layer washed with 5% aq.acetic acid solution (50 mL), 5% aq.NaHCO3 solution (100 mL) and 10% aq.NaCl solution (50 mL). The organic layer was evaporated under vacuum at 35° C. 1,4 dioxane (15 mL) was added to the reaction mass at 50° C. The organic layer was evaporated under vacuum at 50° C. 1,4 dioxane (50 mL) was added to the resulted reaction mass at 50° C. The reaction mass was heated to 75° C. The reaction mass was stirred for 10 minutes at 75° C. The reaction was seeded with Venetoclax (0.025 g) at 55° C. The reaction mass was cooled to 28° C. and stirred for 9 hrs. The reaction mass was filtered and washed with 1,4-dioxane (20 mL). The resulted solid and 1,4 dioxane(110 mL) were charged into a round bottom flask at 28° C. The reaction mass was heated to 70° C. The reaction mass was stirred for 35 minutes at 70° C. The reaction mass was seeded with Venetoclax (0.025 g) at 52° C. The reaction mass was cooled to 28° C. and stirred for 11 hrs. The reaction mass was filtered under vacuum and washed with 1,4-dioxane (20 mL). The solid was suck dried for 20 minutes. The solid was dried under vacuum at 60° C. Product weight: 4.62 g; Purity by HPLC: 99.5%; N-Oxide impurity by HPLC: 0.007%

### Example-5

### Preparation of Crystalline Venetoclax

Venetoclax (150 g) and methanol (2250 mL) were charged into a round bottom flask at 28° C. The reaction mass was stirred for 35 minutes at 28° C. The reaction mass was heated to 58° C. and stirred for 3 hrs. The reaction mass was cooled to 28° C. and stirred for 1 hr. The reaction mass was filtered and washed with methanol (750 mL). The resulted solid and water (4500 mL) were charged into a round bottom flask at 28° C. The reaction mass was stirred for 5 minutes at 28° C. The reaction mass was heated to 59° C. and stirred for 2 hrs. The reaction mass was filtered and washed with water (3000 mL). The solid was suck dried for 3 hrs. The solid was dried under vacuum at 70° C. Product weight: 141 g.

### Example-6

### Preparation of Amorphous Form of Venetoclax

Venetoclax (10 g) was dissolved in glacial acetic acid (40 mL) at 55° C. The reaction solution was filtered and washed with acetic acid (10 mL). The reaction solution was added to aqueous ammonia solution (120 mL ammonia in 600 mL water) at 30° C. The reaction mixture was stirred for 2 hrs at 30° C. The resulted suspension was filtered and washed with water (100 mL). The solid and water (200 mL) were charged into a chemglass reactor at 32° C. The reaction mass was stirred for 1 hr at 32° C. The resulted suspension was filtered and washed with water (100 mL). The solid was suck dried for 30 minutes. The solid was dried under vacuum at 70° C. Product weight: 6.65 g.

1. Substantially pure venetoclax, wherein the venetoclax comprises less than 0.01% of N-oxide compound corresponding to Formula (A):

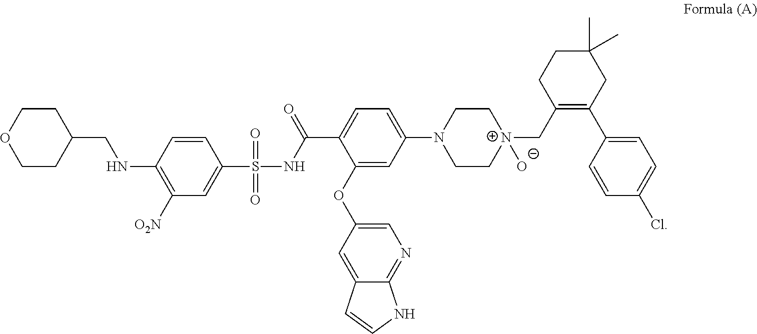 [SMILES: CC1(C)CCC(C[N+]2([O-])CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

2. The substantially pure venetoclax according to claim 1, wherein the venetoclax comprises less than 0.008% of N-oxide compound.

3. A pharmaceutical formulation comprising the substantially pure venetoclax of claim 1 and a pharmaceutically acceptable excipient.

4. A process for the preparation of substantially pure venetoclax of claim 1, the process comprising the steps of:
a) providing venetoclax in ether solvent; and
b) isolating the substantially pure venetoclax.

5. The process of claim 4, wherein the ether solvent is selected from the group consisting of 1,4 dioxane, tetrahydrofuran, and mixture thereof.

6. Amorphous venetoclax in a free drug particulate form.

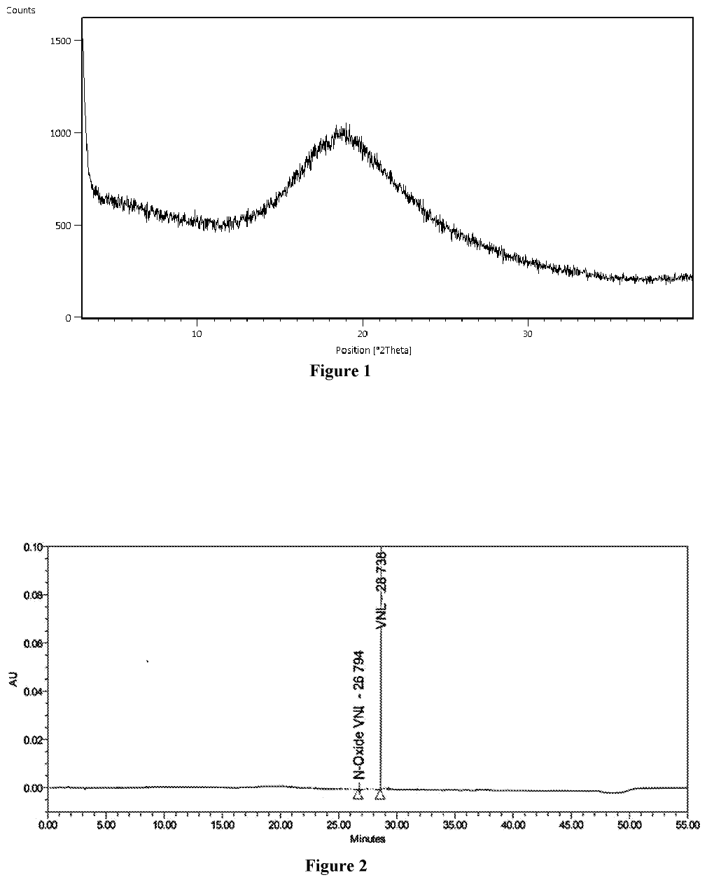

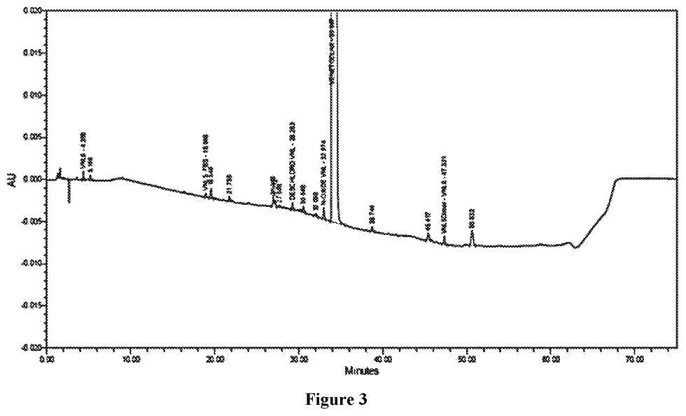

7. The amorphous venetoclax of claim 6, substantially as depicted in FIG. 1.

8. The amorphous venetoclax of claim 6, having carr's index (%) less than 26.

9. The process for the preparation of amorphous venetoclax in a free drug particulate form of claim 6, the process comprising the steps of:
a) providing venetoclax in glacial acetic acid;
b) treating with ammonia solution; and
c) isolating the amorphous form of venetoclax in a free drug particulate form.

10. A pharmaceutical formulation comprising amorphous venetoclax in a free drug particulate form of claim 6 and a pharmaceutically acceptable excipient.
